(19) United States
(12) Patent Application Publication
(10) Pub. No.: US 2013/0204355 A1
(43) Pub. Date: Aug. 8, 2013

(54) MEDICAL DEVICE MADE OF EPTFE PARTIALLY COATED WITH AN ANTIMICROBIAL MATERIAL

(71) Applicants: Helmut Goldmann, Kassel (DE); Dennis Langanke, Kassel (DE); Richard W. Oliver, Westford, MA (US)

(72) Inventors: Helmut Goldmann, Kassel (DE); Dennis Langanke, Kassel (DE); Richard W. Oliver, Westford, MA (US)

(73) Assignees: SPIRE BIOMEDICAL, Bedford, MA (US); AESCULAP AG, Tuttlingen/Donau (DE)

(21) Appl. No.: 13/577,974

(22) PCT Filed: Feb. 11, 2011
(86) PCT No.: PCT/EP2011/052043

§ 371 Date: Oct. 23, 2012

Related U.S. Application Data
(60) Provisional application No. 61/303,990, filed on Feb. 12, 2010.

(51) Int. Cl. A61L 27/28; A61F 2/06

(52) U.S. Cl. CPC A61L 27/28; A61F 2/06
USPC 623/1.39

(57) ABSTRACT

A medical device includes a porous article of expanded polytetrafluoroethylene (ePTFE) having a microstructure of nodes interconnected by fibrils, and a coating including an antimicrobial material at the surface of the porous article, wherein the microstructure at a surface of the porous article is only partially coated with the antimicrobial material.

### RELATED APPLICATIONS

This is a §371 of International Application No. PCT/EP2011/052043, with an international filing date of Feb. 11, 2011 (WO 2011/098565 A1, published Aug. 18, 2011), which is based on U.S. Patent Application No. 61/303,990, filed Feb. 12, 2010, the subject matter of which is incorporated by reference.

### TECHNICAL FIELD

This disclosure relates to a medical device based on a porous article of expanded polytetrafluoroethylene (ePTFE) and a method for manufacturing the medical device.

### BACKGROUND

Microbial colonization and subsequent infections of vascular grafts are a well recognized complication in the field of vascular surgery. In particular, such infections are often associated with a high rate of amputation and mortality.

It is well known to use extruded tube structures of polytetrafluoroethylene (PTFE) as an implantable material as it exhibits superior biocompatibility. Particularly, PTFE tube structures may be used as vascular grafts in the replacement or repair of a blood vessel as PTFE exhibits low thrombogenicity. In vascular applications, the grafts are generally manufactured from expanded polytetrafluoroethylene (ePTFE) tube structures. These tube structures have a microporous microstructure defined by interspaced nodes interconnected by elongated fibrils. On the one hand, this microstructure may be selectively adjusted by appropriate expanding and stretching conditions during the manufacturing of ePTFE tubes to lower the risk of thrombosis and promote tissue ingrowth from the outer periphery of the graft. On the other hand, the microstructure of ePTFE is a labile structure which makes it difficult and complicated to equip ePTFE grafts with antimicrobial properties without destroying the node and fibril microstructure of ePTFE.

Accordingly, it could be helpful to provide a medical device made of ePTFE having antimicrobial properties, in particular long-lasting antimicrobial properties and, additionally, having an intact microstructure of nodes that are interconnected by fibrils. Furthermore, it could be helpful that the medical device, in particular its microstructure, resists mechanical forces that typically occur in a human or animal body without being damaged or destroyed. Also, it could be helpful to provide a medical device comprising a reduced amount of an antimicrobial material and nonetheless having a sufficient antimicrobial effect.

### SUMMARY

We provide a medical device including a porous article of expanded polytetrafluoroethylene (ePTFE) having a microstructure of nodes interconnected by fibrils, and a coating including an antimicrobial material at the surface of the porous article, wherein the microstructure at a surface of the porous article is only partially coated with the antimicrobial material.

We also provide a method of manufacturing the medical device, including a) compressing a porous article of ePTFE having a microstructure of nodes interconnected by fibrils, b) coating the compressed porous article with an antimicrobial material, and c) expanding the coated porous article.

### BRIEF DESCRIPTION OF THE DRAWINGS

FIG. 1 schematically illustrates the node and fibril microstructure of expanded polytetrafluoroethylene (ePTFE).

FIG. 2 schematically illustrates the node and fibril microstructure of ePTFE after compressing the porous article.

FIG. 3 schematically illustrates the node and fibril microstructure of ePTFE after coating the compressed porous article with an antimicrobial material.

FIG. 4 schematically illustrates the coated node and fibril microstructure of ePTFE after expanding the coated porous article.

### DETAILED DESCRIPTION

Our medical device comprises a porous article of expanded polytetrafluoroethylene (ePTFE) having a microstructure of interspaced nodes interconnected by fibrils and having a coating including an antimicrobial material at the surface of the porous article, wherein the microstructure is only partially coated with the antimicrobial material at the surface of the porous article.

The term “polytetrafluoroethylene” as used herein may also comprise a copolymer of tetrafluoroethylene.

The term “copolymer” as used herein preferably means a polymer that comprises at least two different monomer units.

Accordingly, the term “copolymer of tetrafluoroethylene” as used herein encompasses a polymer composed of at least one further monomer unit along tetrafluoroethylene. Such a further monomer unit may be selected from the group consisting of chlorotrifluoroethylene, perfluoroalkoxy-tetrafluoroethylene, hexafluoropropylene, tetrafluoropropylene and combinations thereof.

Preferably, only nodes or node segments at the surface of the porous article are coated, in particular, completely coated with the antimicrobial material. The fibrils of the microstructure, in particular fibrils at the surface of the porous article, may be at most partially coated with the antimicrobial material. More preferably, the fibrils of the microstructure, in particular fibrils at the surface of the porous article, are free of a coating including an antimicrobial material. Coating or predominantly coating of the fibrils would lead to an undesired stiffening and undesired brittleness of the fibrils being more susceptible to breakage under mechanical forces that typically appear when the medical device is, for example, bent, stretched or compressed.

Preferably, the nodes present at the surface of the porous article are coated with the antimicrobial material and the fibrils of the microstructure, in particular at the surface of the porous article, are free of a coating including an antimicrobial material. Such a device advantageously resists more easily mechanical forces, in particular bending forces, expanding forces and compressing forces, that may typically occur in a patient's body without damaging or destroying the microstructure. Furthermore, it is possible to provide a medical device having alternating antimicrobial properties at its surface (preferably nodes coated and interconnecting fibrils uncoated). Nonetheless, the surface may largely exercise an antimicrobial effect, in particular a long-lasting antimicrobial effect, sufficient to reduce the risk of post-surgical infections.

Preferably, the coating including an antimicrobial material leaves internodal spaces open of the microstructure, in particular at the surface of the porous article. Thus, natural tissue ingrowth is possible into the article from its outer periphery leading to a secure anchoring of the article within the body of a human or animal patient and facilitating a rebuilding of the original anatomical circumstances around the implantation region. The term “internodal space” as used herein refers to the space between the node surfaces spanned by the fibrils.

Generally, the nodes of the microstructure may have an elongate, in particular an ellipsoidal, shape. More preferably, the nodes have a relatively uniform shape and in particular uniform size. The nodular size, in particular the nodular length, may be 1 to 800 μm, in particular 5 to 500 μm, and preferably 10 to 300 μm. The interconnecting fibrils may have a bent, wavy or straight appearance. More preferably, the fibrils of the microstructure that interconnect nodes may have a straight and in particular parallel appearance. Further, the fibrils may have a length of 1 to 150 μm, in particular 20 to 80 μm. Furthermore, the fibrils may have a diameter of 0.1 to 10 μm, in particular 0.5 to 5 μm. Typically, the microstructure may have internodal spaces of 1 to 150 μm, in particular 10 to 100 μm, and preferably 20 to 80 μm.

Further, the coating including an antimicrobial material may have a proportion of 0.01 to 5.0% by weight, in particular 0.1 to 3.0% by weight, and preferably 0.3 to 1.5% by weight, related to the total weight of the porous article. Furthermore, the coating including an antimicrobial material, taken at the thickness, in particular wall thickness, of the porous article may have a proportion of 0.001 to 0.2%, in particular 0.004 to 0.125%, and preferably 0.01 to 0.04%. The coating including an antimicrobial material may be arranged in a depth of 1 to 150 μm, in particular 20 to 100 μm, preferably 40 to 80 μm, taken at the surface of the porous article.

Preferably, the coating including an antimicrobial material is of such thickness that in vivo, i.e., after implantation, it has a dwell time on the article's surface of more than 1 month, particularly more than 6 months, and releases the antimicrobial material during this time. Preferably, the coating including an antimicrobial material has a layer thickness of 100 to 4000 Å (angstrom), in particular 400 to 2500 Å (angstrom), and preferably 800 to 1600 Å (angstrom).

The microstructure may include nodes arranged in differing internodal spaces. Preferably, the internodal spaces are arranged in a gradient. More preferably, the internodal spaces of the microstructure increase, in particular gradually increase, from one surface of the porous article to an opposing surface. It is especially preferred that the internodal spaces increase, in particular gradually increase, from an inside surface to an outside surface of the porous article. It is further preferred that internodal spaces are sufficiently large enough at one surface, in particular at an outside surface, of the porous article, to allow natural tissue ingrowth into the article from its outer periphery. In contrast, it is especially preferred that internodal spaces are sufficiently small enough at a surface opposing the afore-mentioned one surface, preferably at an inside surface of the article, that substantially prevents natural tissue ingrowth or platelet activation and platelet adhesion but may facilitate the passage of low molecular compounds, for example, nutrients, biological agents, medicinal agents, et cetera.

The coating may further substantially consist of the antimicrobial material. However, the coating may include minor amounts of additional components such as pharmaceutical agents, antimicrobial agents, disinfectants, biological agents, radiopaque materials, et cetera.

The antimicrobial material may be selected from the group consisting of an antimicrobial metal, antimicrobial alloy, antimicrobial compound, antimicrobial salt, in particular oxide, and combinations thereof. Antimicrobial metals, antimicrobial alloys and/or anitmicrobial metal compounds, in particular antimicrobial metal salts, are especially preferred. The antimicrobial material may be selected from the group consisting of zirconium, copper, zinc, silver, gold, palladium, platinum, iridium, aluminium, nickel, tungsten, molybdenum, tantalum, titanium, iodine, alloys thereof, compounds thereof, salts thereof, for instance oxides thereof, and combinations thereof. Silver is especially preferred. As used herein, the term “antimicrobial material” refers to a material which inhibits the growth of micro-organisms such as pathogenic bacteria, protests and/or fungi which may cause serious infections within a patient.

The porous article may comprise an adhesion-promoting agent. Typically, the adhesion-promoting agent is adjacent to the surface of the porous article. Preferably, the adhesion-promoting agent is arranged in the form of a layer. Further, the adhesion-promoting agent may be a metal, in particular titanium. The porous article may further comprise a species, in particular a layer thereof, which forms a barrier to degradation or diffusion of an adhesion-promoting agent and, for instance, impede galvanic interaction between the antimicrobial material and an adhesion-promoting agent. The species may be a metal, in particular palladium. Preferably, the barrier species is adjacent to the surface of the porous article and is in particular interposed between an adhesion-promoting agent and the antimicrobial material.

The porous article may comprise a leak-proofing coating or impregnation. Such a coating and impregnation, respectively, is typically designed on the surface of the article. Generally, such a coating and impregnation, respectively, seals internodal spaces of the microstructure at the surface of the porous article. Suitable materials for the coating and impregnation, respectively, may be biological polymers and/or synthetic polymers, in particular copolymers. Synthetic polymers, in particular copolymers, based on hydroxyl acids are especially preferred. Suitable materials may be selected from the group consisting of collagen, gelatine, albumin, carboxymethylcellulose, polyvinylalcohol, polylactide, polyglycolide, poly-ε-caprolactone, poly-trimethylencarbonate, poly-para-dioxanone, copolymers thereof and combinations thereof. By suitable choice of the polymers, the desired duration of absorption may be set. This is preferably within four months and in particular within 40 days. Such a time is normally expedient since depending on the type of the medical device, the impregnation action is no longer necessary during this time because of the incurring connective tissue.

Preferably, the coating including an antimicrobial material is deposited onto the surface of the porous article by a surface treatment, in particular by a dry coating process. More preferably, the coating including an antimicrobial material is vapor deposited, in particular ion-beam-assisted deposited, onto the surface of the porous article.

Preferably, the porous article is a tubular article, typically a hollow tubular article. In other words, the porous article is preferably designed as a tubing, typically having a lumen encased by the tubing wall. Preferably, the coating including an antimicrobial material is present at the outside surface of the tubular article. In particular, only nodes or node segments at the outside surface of the tubular article are coated, in particular completely coated, with the antimicrobial material. The fibrils of the microstructure, in particular fibrils at the outside surface of the tubular article, are free of a coating including an antimicrobial material. Internodal spaces, in particular at the outside surface of the tubular article, are preferably free of a coating including an antimicrobial material.

Furthermore, the tubular article may have different pore sizes, particularly internodal spaces, at its inside and outside surface. Preferably, the inside surface of the tubular article is basically arranged smooth. In other words, the microstructure at the inside surface of the tubular article preferably offers no or only a negligible resistance to flow of blood and, consequently, platelet activation and platelet adhesion may be reduced. Furthermore, it is preferred that the inside surface may have a pore structure, in particular internodal spaces, facilitating the passage of small molecular compounds, in particular of nutrients, biological agents and/or medical agents, through the wall of the tubular article into the lumen thereof. Thus, a nutrition supply to a neointima is possible that preferably lines the insides surface of the tubular article. It is thus possible to greatly reduce calcification of neointima that may result from nutritional deficiency.

Further, biological and/or medical agents may enter the lumen of the tubular article and, for instance, may help to prevent the manifestation of a thrombosis. Preferably, the microstructure at the outside surface of the tubular article promotes tissue ingrowth, in particular ingrowth of fibroblasts, from the outer periphery of the article. This contributes to a secure anchoring of the article with surrounding body tissue. Furthermore, the aforementioned nutrient supply is essentially based on capillaries which densely develop on fully grown fibroblasts. Preferably, pore sizes, in particular internodal spaces, of the microstructure increase, particularly gradually increase, from the inside surface to the outside surface of the tubular article.

The porous article may be designed as a tubular article having an internal diameter of 2 to 50 mm.

The porous article, in particular a tubular porous article, may have a thickness, in particular a wall thickness, of 0.1 to 1.0 mm, in particular 0.25 to 0.75 mm, preferably 0.4 to 0.6 mm.

The porous article may be designed as a two-dimensionally shaped (planar) article, in particular as a mesh, sling, patch and the like. Accordingly, the medical device may be designed as a hernia mesh, prolaps mesh or aconuresis sling.

Preferably, the porous article is a tubular prosthesis. Preferably, the porous article is an arterial prosthesis or a vein prosthesis. More preferably, the porous article is an arterial prosthesis.

Further, the medical device may be on hand in a sterile form and in particular tailored form.

We further provide a method for the manufacture of a medical device comprising the steps of:
a) compressing a porous article, preferably a tubular porous article, of expanded polytetrafluoroethylene (ePTFE) having a microstructure of interspaced nodes interconnected by fibrils,
b) coating the compressed porous article, preferably tubular porous article, with an antimicrobial material, and
c) expanding the coated porous article, preferably tubular porous article.

Due to the compressing step a), the microstructure of the article is also compressed, i.e., the nodes are transferred into a compact state minimizing and in particular removing internodal spaces. Thus, basically only the nodes at the surface of the porous article are coated with the antimicrobial material. The fibrils of the microstructure are basically not coated with the antimicrobial material. As already mentioned, coated fibrils have an increased stiffness and brittleness thus being more susceptible to breakage under mechanical forces leading at least to a partially destruction of the node and fibril microstructure. The method facilitates a partial coating of the porous article maintaining the original node and fibril microstructure of the porous article. This effects unimpaired flexibility of the porous article and the medical device, respectively, and a better resistance to mechanical forces typically occurring in a patient's body.

Preferably, the porous article is compressed on a mandrel, in particular on a rotatable mandrel. With the rotatable mandrel, an equally coating of the porous article with the antimicrobial material is possible. Preferably, the mandrel is designed as a rod. Furthermore, the mandrel may be manufactured from metal, steel or polymer, in particular polyethylene or polyvinylalcohol. In particular, the mandrel may have a diameter, in particular a radial diameter of 2 to 50 mm.

The compressed porous article may be coated with the antimicrobial material by a dry coating process, in particular by a physical vapor deposition process (PVD process), preferably an ion-beam-assisted deposition process (IBAD process).

In general, ion-beam processes are low-temperature, high-technology processes with excellent quality control to achieve good adherence, ductility, reproducibility, reliability and thickness of deposition control at a high throughput and with no chemical residues, thus being both environmentally and occupationally a safe dependable technique. Typically, an ion-beam-assisted coating apparatus comprises a vacuum chamber system formed of a low-vacuum antechamber and a high-vacuum processing chamber, air-tightly separated from each other by a gate movable between an opened position and a closed position. An ion source, which can be a bucket-type ion source is mounted within the high-vacuum processing chamber, normally in a position diametrically opposed to the low-vacuum antechamber. The ion source is typically fed by one or more gases such as argon, oxygen, neon, and/or helium, from a suitable gas supply source via a mass flow controller, regulating the rate of gas feed.

Further, an evaporator is also mounted in the high-vacuum processing chamber, normally in operative association with the ion source. The evaporator is designed to vaporize particular evaporants, more specifically metallic evaporants to dry-coat a specific substrate, in particular a medical device, therewith, being assisted in the dry-coating by an ion-beam emanating from the ion source. Suitable evaporants include zirconium, copper, zinc, silver, gold, palladium, platinum, iridium, aluminium, nickel, tungsten, molybdenum, tantalum, titanium, and their respective alloys, salts and compounds. Normally, a vapor shutter, designed to be rotated in and out of place of the evaporator, shields the substrate from the evaporants when in place. The substrate to be dry-coated is normally introduced into the vacuum chamber system with the aid of a suitable substrate holder. Preferably, the substrate holder is mounted for both rotational and translatory motion on a shaft and is introduced in the antechamber through a hinge-like mounted end-blade.

With respect to the above-made comments, the IBAD process is typically performed in a suitable vacuum chamber system including a processing chamber. The required vacuum environment is normally created by a vacuum. Preferably, the ion-beam-assisted deposition is performed under a vacuum pressure of at least 10⁻⁴ torr, in particular of at least 10⁻⁵ torr. Further, the ion-beam-assisted deposition may be performed at a temperature <150° C. Moreover, the porous article may be exposed to an ion-beam energy of 5 to 10000 eV, in particular 50 to 5000 eV, and preferably 200 to 1000 eV. The above-mentioned ion-beam energy is preferably intended to achieve an ion-beam current density on the surface of the porous article of 0.1 to 500 μA/cm², in particular 1 to 250 μA/cm², preferably 10 to 80 μA/cm². The ion-beam-assisted deposition may be performed during a time period of 0.1 to 500 minutes, in particular 1 to 200 minutes, preferably 5 to 75 minutes. Further, the ion-beam-assisted deposition may be performed applying a deposition rate of 0.1 to 50 Å (angstrom)/sec, in particular 1 to 25 Å (angstrom)/sec, and preferably 2 to 10 Å (angstrom)/sec.

Preferably, the ion-beam-assisted deposition is carried out under the following process parameters: a vacuum pressure of at least 10⁻⁵ torr, an ion-beam energy of 200 eV to 1000 eV, an ion-beam current density of 10 to 80 μA/cm², and a deposition rate to 2 to 10 Å (angstrom)/sec.

It is further possible to coat the compressed porous article with the antimicrobial material by dipping, immersing, spraying, brushing and/or painting techniques. In particular, the compressed porous article may be coated by wet-chemical procedures. For example, the compressed porous article to be coated may be immersed or dipped into a liquid dispersion, suspension or solution of the antimicrobial material. Galvanic techniques are in principal also possible. Because of the compressing step a) preceding the coating step c), solvents, reagents, additives and the like typically employed in the techniques described above cannot penetrate the node and fibril microstructure that may possibly lead to undesired residues in the article and medical device, respectively.

The coated porous article may be expanded subsequent to a purification step, in particular rinsing or washing step, of the article. Thus, laborious and time-consuming purifications of the node and fibril microstructure from solvents, reagents, et cetera may be circumvented. Additionally, the risk of harmful residues being entrapped by the node and fibril microstructure can be lowered. Altogether, this leads to an increased biocompatibility of the porous article and the medical device, respectively.

We further provide a medical device, preferably a vascular prosthesis, obtained or obtainable according to one of the afore-described methods.

Our devices and methods will be illustrated in more detail by preferred examples presented in the figures and description of the figures. Individual features may be realized exclusively or in combination with other features. Any described example is given for the sole purpose of illustration and better understanding, and is no way to be interpreted as a limitation.

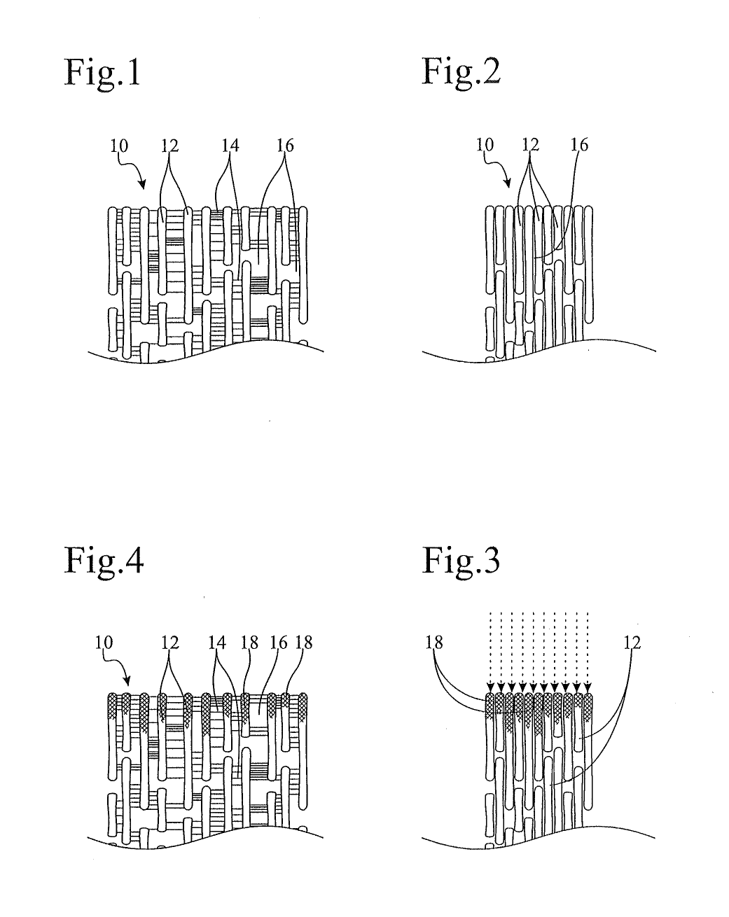

FIG. 1 schematically illustrates the node and fibril microstructure 10 of expanded polytetrafluoroethylene (ePTFE) of a porous article before compression. The microstructure 10 comprises interspaced nodes 12 that have typically an elongate shape being interconnected by fine fibrils 14. Dependent on the stretching and expanding conditions during the manufacturing process of ePTFE the form of the nodes 12 and in particular the appearance of the interconnecting fibrils 14 may vary. For example, the fibrils 14 may be bent, wavy or, as illustrated in FIG. 1, may have a straight and particularly parallel appearance.

FIG. 2 schematically illustrates the node and fibril microstructure 10 of ePTFE after compressing a porous article. Due to the compressing step the nodes 12 are packaged densely minimizing the internodal spaces 16. Preferably, the nodes 12 are basically not interspaced any longer by the fibrils 14. Insofar, the originally open node and fibril microstructure 10 as illustrated in FIG. 1 is transferred into a closed microstructure that is based on densely packaged nodes as illustrated in FIG. 2.

FIG. 3 schematically illustrates the node and fibril microstructure 10 of ePTFE after coating a porous article, but before expanding the article (the coating process is depicted by dotted arrows). Basically, only nodes 12 and segments thereof, respectively, at the surface of the porous article are coated with an antimicrobial material 18. The fibrils 14 at the surface are basically uncoated that is mainly due to their compression between the nodes 12 and their shielding that is associated with the compact configuration of the nodes 12. The coating 18 per se may be arranged in a certain depth, taken from the surface of the porous article.

FIG. 4 schematically illustrates the node and fibril microstructure 10 of ePTFE of a porous article. At the surface of the article, basically only nodes 12 and segments thereof, respectively, of the node and fibril microstructure 10 are coated with the antimicrobial material 18. Interconnecting fibrils 14 at the surface of the porous article are free of a coating including an antimicrobial material 18. Further, internodal spaces 16 at the surface of the porous article are also free of a coating including an antimicrobial material 18. Altogether, the porous article comprises a surface being only partially coated with the antimicrobial material 18. Nonetheless, coating the nodes 12 at the surface of the porous article is sufficient for a preferably long-term antimicrobial action of the article. Furthermore, the porous article comprises an unimpaired node and fibril microstructure 10 that is advantageous regarding tissue ingrowth into the article from its outer periphery and the prophylaxis of post-surgical complications like the formation of thrombosis and restenosis of a tubular article, preferably a vascular prosthesis.

### EXAMPLES

Prostheses of ePTFE are compressed on mandrels before clamped in a rotatable clamp device so that they hang freely as a bundle of parallel tubes with spaces between them. The clamp device is introduced into a vacuum chamber suitable for carrying out an ion-beam-assisted deposition technique (IBAD technique), the ePTFE prostheses being vapor-deposited with silver and at the same time bombarded with argon ions. The coating operation is conducted until a silver layer thickness of 1300 Å is reached on the outside of the ePTFE prosthesis or the nodes located there. If desired, a primary coating can be effected by vapor-deposition of other metals.

After the coating process the ePTFE prostheses are expanded. The ePTFE prostheses with an internal diameter of 8 mm and a wall thickness of 500 μm coated in this way each has a segment at the outer surface which is only partially coated with silver (basically only nodes or node segments at the outside surface of the prostheses are coated with silver). The silver coating has a film thickness of 1300 Å resulting in a proportion of silver relative to the total weight in the range of 0.3 to 0.5% by weight which is equivalent to a proportion of 0.01 to 0.04% in relation to the wall thickness of the porous ePTFE prostheses. The coating is applied to the ePTFE prostheses in a depth of 40 to 80 μm.

1. A medical device comprising:
a porous article of expanded polytetrafluoro-ethylene (ePTFE) having a microstructure of nodes interconnected by fibrils, and
a coating including an antimicrobial material at the surface of the porous article, wherein the microstructure at a surface of the porous article is only partially coated with the antimicrobial material.

2. The medical device according to claim 1, wherein only nodes or segments thereof at the surface of the porous article are coated with the antimicrobial material.

3. The medical device according to claim 1, wherein fibrils at the surface of the porous article are at most partially coated with the antimicrobial material.

4. The medical device according to claim 1, wherein the coating including an antimicrobial material leaves internodal spaces open at the surface of the porous article.

5. The medical device according to claim 1, wherein the coating including an antimicrobial material has a proportion of 0.01 to 5.0% by weight, relating to the total weight of the porous article.

6. The medical device according to claim 1, wherein the coating has a layer thickness of 100 to 4000 Å.

7. The medical device according to claim 1, wherein the antimicrobial material is selected from the group consisting of an anti-microbial metal, antimicrobial alloy, antimicrobial compound, salts thereof and combinations thereof.

8. The medical device according to claim 1, wherein the antimicrobial material is selected from the group consisting of zirconium, copper, zinc, silver, gold, palladium, platinum, iridium, aluminium, nickel, tungsten, molybdenum, tantalum, titanium, iodine, alloys thereof, compounds thereof, salts thereof and combinations thereof.

9. The medical device according to claim 1, wherein the porous article is a tubular article.

10. The medical device according to claim 9, wherein only nodes at an outside surface of the tubular article are coated with the antimicrobial material.

11. The medical device according to claim 1, which is a tubular prosthesis.

12. A method of manufacturing a medical device according to claim 1, comprising:
a) compressing a porous article of ePTFE having a microstructure of nodes interconnected by fibrils,
b) coating the compressed porous article with an antimicrobial material, and
c) expanding the coated porous article.

13. The method according to claim 12, wherein the porous article is compressed on a rotatable mandrel.

14. The method according to claim 12, wherein the compressed porous article is coated by a physical vapour deposition or an ion-beam-assisted deposition.

15. (canceled)
